(19) United States
(12) Patent Application Publication
(10) Pub. No.: US 2020/0323763 A1
(43) Pub. Date: Oct. 15, 2020

(54) THICKENED ONE-COMPONENT HAIR TONER

(71) Applicant: Henkel AG & Co. KGaA, Duesseldorf (DE)

(72) Inventors: JUERGEN SCHOEPGENS, Schwalmtal (DE); TORSTEN LECHNER, Langenfeld (DE)

(73) Assignee: Henkel AG & Co. KGaA, Duesseldorf (DE)

(21) Appl. No.: 16/765,428

(22) PCT Filed: Oct. 16, 2018
(86) PCT No.: PCT/EP2018/078134

§ 371 Date: May 19, 2020

(30) Foreign Application Priority Data
Dec. 15, 2017 (DE) 10 2017 222 955.1

(51) Int. Cl. A61K 8/81; A61K 8/06; A61K 8/04; A61K 8/92; A61K 8/34; A61Q 5/06

(52) U.S. Cl. CPC A61K 8/8147; A61K 8/064; A61K 8/042; A61K 8/92; A61K 2800/48; A61K 8/8152; A61Q 5/065; A61K 2800/432; A61K 8/342

(57) ABSTRACT

The present disclosure relates to a non-oxidative agent for dyeing keratin fibers, in particular human hair, containing in a cosmetic carrier at least one direct dye and sodium polyacrylate. Furthermore, the present disclosure relates to a method for non-oxidative, preferably semi-permanent, coloring of keratin fibers, in particular human hair, using the agent as contemplated herein.

### CROSS-REFERENCE TO RELATED APPLICATION

This application is a U.S. National-Stage entry under 35 U.S.C. § 371 based on International Application No. PCT/EP2018/078134, filed Oct. 16, 2018, which was published under PCT Article 21(2) and which claims priority to German Application No. 10 2017 222 955.1, filed Dec. 15, 2017, which are all hereby incorporated in their entirety by reference.

### TECHNICAL FIELD

The present application relates to an agent for dyeing keratin fibers, in particular human hair, and a method for non-oxidative, preferably semi-permanent, dyeing of keratin fibers, in particular human hair.

### BACKGROUND

The change in shape and color of keratin fibers, especially hair, is an important area of modern cosmetics. As a result, the appearance of the hair can be adapted both to current fashion trends and to the individual wishes of the individual. To change the hair color, the expert knows various coloring systems depending on coloring requirements. Oxidation dyes are usually used for permanent, intensive dyeing with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by excellent, long-lasting dyeing results, but are also associated with a certain degree of hair damage.

If the user wants to reduce hair damage or only change the hair color temporarily, he can use colorants that are based on direct dyes. Here, already fully formed dyes from the dye diffuse into the hair fiber. Compared to oxidative hair dyeing, the dyeing obtained with direct dyes have a shorter shelf life and quicker wash ability. It is advantageous, to dye hair with direct dye for reduced hair damage.

The aforementioned direct dyeing dyes are usually incorporated into a cosmetically suitable carrier, such as a cream. The carrier ensures a homogeneous distribution and a sufficient residence time of the hair dye on the hair.

A disadvantage is the complex production of such a cream. A lot of energy is required to melt the fat components and emulsify them. The subsequent cooling process consumes large amounts of cooling water.

Due to the high fat content in conventional creams, continuous production is difficult to achieve. The components in the melt must be added to each other to ensure that they can be pumped.

### BRIEF SUMMARY

In certain embodiments, a non-oxidative agent is provided for dyeing keratin fibers and is included in a cosmetic carrier. The agent comprises at least one substantive dye; and sodium polyacrylate.

In other embodiments, a process for the non-oxidative coloring of keratin fibers is provided. The process includes applying an agent directly to the keratin fibers in the form of a one-component agent. The agent comprises at least one substantive dye; and sodium polyacrylate.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The present disclosure was based on the object of providing a non-oxidative agent for dyeing keratin fibers, in particular human hair, which can be produced under the most economical and sustainable conditions possible. Furthermore, the present disclosure was based on the object of providing a features for the non-permanent dyeing of keratin fibers, in particular human hair, which can be packaged under the most economical and sustainable conditions possible. Furthermore, the present disclosure was based on the object of providing a features for the non-permanent dyeing of keratin fibers, in particular human hair, which can be produced continuously.

Furthermore, the non-oxidative agent for the non-permanent dyeing of keratin fibers, in particular human hair, should have good durability on the keratin fibers for a non-oxidative, non-permanent dye even after several washes, simple application and generally short Mark exposure times and good optical results, e.g. in terms of dyeing performance and fastness properties.

These tasks are solved by non-oxidative agents for dyeing keratin fibers, in particular human hair, contained in a cosmetic carrier
(a) at least one substantive dye and
(b) Sodium polyacrylate, preferably with a weight average molecular weight M_{w} in the range from about 1,000,000 to about 20,000,000 Daltons, preferably from about 6,000,000 to about 15,000,000 Daltons.

Keratinous fibers, keratin containing fibers or keratin fibers are to be understood as furs, wool, feathers and in particular human hair. Although the agents as contemplated herein are primarily suitable for lightening and dyeing keratin fibers, in principle there is nothing to prevent their use in other areas.

As contemplated herein is the presence of sodium polyacrylate in addition to the substantive dye is characteristic of the agent. The above-mentioned objects are achieved with the agent as contemplated herein, in particular in agents as contemplated herein in the form of a cream, an emulsion or a gel.

Unless otherwise stated, all information on physical states in the present application relates to a temperature of 20° C. and a pressure of 1013 mbar.

### (a) At Least One Direct Dye

Direct dyes are dyes that are applied directly to the hair and do not require an oxidative process to develop the color. Direct dyes are typically nitro phenylenediamines, nitroaminophenols, azo dyes, anthraquinones or indophenols.

Direct dyes can be divided into anionic, cationic and non-ionic direct dyes.

The terms anionic dye and acid dye are used synonymously in the sense of this present disclosure. Anionic dyes or acid dyes are to be understood as direct dyes which have at least one carboxylic acid group (—COOH) and/or at least one sulfonic acid group (—SO3H). Depending on the pH value, the protonated forms (—COOH, —SO3H) of the carboxylic acid or sulfonic acid groups are present in equilibrium with their deprotonated forms (—COO<→>, —SO3<→> before). The proportion of protonated forms increases with decreasing pH. If direct dyes are used in the form of their salts, the carboxylic acid groups or sulfonic acid groups are in deprotonated form and are neutralized with appropriate stoichiometric equivalents of cations (such as, for example, Na cation or K cations) to maintain electro neutrality. An anionic dye carries no cationic charges.

Dyes that carry only cationic charges are usually referred to as basic dyes. For dyes that only carry anionic charges, the expert speaks of acid dyes.

Suitable acid dyes or anionic direct dyes are selected from tetra bromophenol blue (CAS No. 4430-25-5), Acid Yellow 1 (D&C Yellow 7, Citronin A, Ext. D&C Yellow No. 7, Japan Yellow 403 CI 10316, COLIPA n^{o} B001), Acid Yellow 3 (COLIPA n^{o}: C 54, D&C Yellow N^{o} 10, Quinoline Yellow, E104, Food Yellow 13), Acid Yellow 9 (CI 13015), Acid Yellow 17 (CI 18965), Acid Yellow 23 (COLIPA n^{o} C. 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA n^{o} C015), Acid Orange 10 (C.I. 16230; Orange G sodium salt), Acid Orange 11 (CI 45370), Acid Orange 15 (CI 50120), Acid Orange 20 (CI 14600), Acid Orange 24 (Brown 1; CI 20170; KATSU201; Brown No. 201; Resorcin Brown; Acid Orange 24; Japan Brown 201; D & C Brown No. 1), Acid Red 14 (C.I. 14720), Acid Red 18 (E124, Red 18; CI 16255), Acid Red 27 (E123, CI 16185, C-Rot 46, Echtrot D, FD&C Red Nr.2, Food Red 9, Naphthol red S), Acid Red 33 (Red 33, Fuchsia Red, D&C Red 33, CI 17200), Acid Red 35 (C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red n^{o} 106 Pontacyl Brilliant Pink), Acid Red 73 (CI 27290), Acid Red 87 (Eosin, CI 45380), Acid Red 95 (CI 45425, Erythrosine, Simacid Erythrosine Y), Acid Red 184 (CI 15685), Acid Red 195, Acid Violet 43 (Jarocol Violet 43, Ext. D&C Violet n^{o} 2, C.I. 60730, COLIPA n^{o} C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent Blue AE, Amido Blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant acid green BS, C.I. 44090, Acid Brilliant Green BS, E 142), Acid Black 1 (Black n^{o} 401, Naphthalene Black 10B, Amido Black 10B, CI 20 470, COLIPA n^{o} B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and D&C Brown 1 as well as mixtures of these dyes.

Preferred anionic direct dyes are those under the names Acid Yellow 1, Acid Yellow 3, Acid Yellow 9, Acid Yellow 17, Acid Yellow 23, Acid Yellow 36, Acid Yellow 121, Acid Orange 6, Acid Orange 7, Acid Orange 10, Acid Orange 11, Acid Orange 15, Acid Orange 20, Acid Orange 24, Acid Red 14, Acid Red 18, Acid Red 27, Acid Red 33, Acid Red 35, Acid Red 51, Acid Red 52, Acid Red 73, Acid Red 87, Acid Red 95, Acid Red 184, Acid Red 195, Acid Violet 43, Acid Violet 49, Acid Violet 50, Acid Blue 1, Acid Blue 3, Acid Blue 7, Acid Blue 104, Acid Blue 9, Acid Blue 62, Acid Blue 74, Acid Blue 80, Acid Green 3, Acid Green 5, Acid Green 9, Acid Green 22, Acid Green 25, Acid Green 50, Acid Black 1, Acid Black 52, Food Yellow 8, Food Blue 5, D&C Yellow 7, D&C Yellow 8, D&C Orange 4, D&C Green 5, D&C Orange 10, D&C Orange 11, D&C Red 21, D&C Red 27, D&C Red 33, D&C Violet 2 and/or D&C Brown 1 and tetra bromophenol blue known compounds and mixtures of these dyes.

Preferred cationic substantive dyes are cationic triphenylmethane dyes, such as Basic Blue 7, Basic Blue 26, Basic Violet 2 and Basic Violet 14, and aromatic systems which are substituted with a quaternary nitrogen group, such as Basic Yellow 57, Basic Red 76, Basic Blue 99, Basic Brown 16 and Basic Brown 17 and HC Blue 16, as well as Basic Yellow 87, Basic Orange 31 and Basic Red 51.

Preferred non-ionic direct dyes are selected from HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and 1.4-diamino-2-nitrobenzene, 2-amino-4-nitrophenol, 1.4-bis-(2-hydroxyethyl)amino-2-nitrobenzene, 3-nitro-4-(2-hydroxyethyl)aminophenol, 2-(2-hydroxyethyl)amino-4,6-dinitrophenol, 4-[2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1.4-naphthoquinone, picramic acid and its salts, 2-amino-6-chloro-4-nitrophenol, 4-ethylamino-3-nitrobenzoic acid and 2-chloro-6-ethylamino-4-nitrophenol and mixtures thereof.

Furthermore, naturally occurring dyes, such as those contained in henna red, henna neutral, henna black, chamomile flowers, sandalwood, black tea, walnut, rotten bark, sage, blue wood, madder root, catechu and alkanna root can also be used as direct dyes.

The agent as contemplated herein preferably contains at least one substantive dye in a total amount of from about 0.001-7% by weight, preferably from about 0.01-5% by weight, preferably from about 0.1-4% by weight, in particular from about 0.2-3.5% by weight, extremely preferably from about 0.3-3% by weight, further highly preferable from about 0.5-2% by weight, further extremely preferable from about 0.7-1.2% by weight, in each case based on the weight of the agent as contemplated herein.

### (b) Sodium Polyacrylate

An essential feature of the agents as contemplated herein is the presence of sodium polyacrylate. As contemplated herein, sodium polyacrylate is preferably understood to mean polymers with the CAS number 9003-04-7. Sodium polyacrylates preferred as contemplated herein have a weight-average molecular weight M_{w} in the range from about 1,000,000 to about 20,000,000 daltons, preferably from about 6,000,000 to about 15,000,000 daltons. The average molecular weight Mw can be determined by gel permeation chromatography (GPC) with polystyrene as the internal standard in accordance with DIN 55672-3, Version August 2007,

The sodium polyacrylate leads to a thickening of the agent, which at the same time maintains the consistency of a creamy gel.

In a preferred embodiment, the sodium polyacrylate is contained as sodium polyacrylate pre-gelled in a water-in-oil emulsion. It is particularly preferred here that the water-in-oil emulsion containing sodium polyacrylate, based in each case on its total weight (from about 100% by weight), contains from about 40-60% by weight sodium polyacrylate, a total of from about 25-45% by weight oil(s), a total of from about 0.5-4.9% by weight surfactant(s) and from about 0.5-4.9% by weight water.

The oil contained in the water-in-oil emulsion containing sodium polyacrylate is particularly preferably selected from natural and synthetic hydrocarbons, particularly preferably from mineral oil, paraffin oils, C₁₈-C₃₀ isoparaffins, in particular isoeicosane, polyisobutenes and polydecenes, C₈-C₁₆ isoparaffins, as well as 1,3-di-(2-ethylhexyl)cyclohexane; the benzoic acid esters of linear or branched C₈-C₂₂ alkanols; triglycerides of linear or branched, saturated or unsaturated, optionally hydroxylated C₈₋₃₀ fatty acids, in particular natural oils; the dicarboxylic acid esters of linear or branched C₂-C₁₀-alkanols; the esters of linear or branched saturated or unsaturated fatty alcohols with 2 to 30 carbon atoms with linear or branched saturated or unsaturated fatty acids with 2 to 30 carbon atoms, which may be hydroxylated; the adducts of 1 to 5 propylene oxide units with mono- or polyvalent C₈-C₂₂ alkanols; the C₈-C₂₂ fatty alcohol esters of monovalent or polyvalent C₂-C₇ hydroxycarboxylic acids; the symmetrical, asymmetrical or cyclic esters of carbonic acid with C₃-C₂₂ alkanols, C₃-C₂₂ alkanediols or C₃-C₂₂ alkanetriols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols; silicone oils and mixtures of the aforementioned substances. Particularly preferred oil as contemplated herein is mineral oil.

The surfactant contained in the water-in-oil emulsion containing sodium polyacrylate is particularly preferably selected from non-ionic surfactants. Non-ionic surfactants used with particular preference are selected from 7-80 moles of ethylene oxide per mole of ethoxylated castor oil, ethoxylated C₈-C₂₄ alkanols with 5-30 moles of ethylene oxide per mole, ethoxylated C₈-C₂₄ carboxylic acids with 5-30 moles of ethylene oxide per mole, with 4-50 moles of ethylene oxide per mole of ethoxylated sorbitan monoesters of linear saturated and unsaturated C₁₂-C₃₀ carboxylic acids, which can be hydroxylated, especially those of myristic acid, palmitic acid, stearic acid or of mixtures of these fatty acids, alkyl mono- and oligoglycosides with 8 to 22 carbon atoms in the alkyl radical and their ethoxylated analogues, and mixtures of the aforementioned substances.

The ethoxylated C₈-C₂₄ alkanols have the formula R¹O(CH₂CH₂O)ₙH, where R¹ stands for a linear or branched alkyl and/or alkenyl radical with 8-24 carbon atoms and n, the average number of ethylene oxide units per molecule, is a number from 5 to 30, preferably 6 to 20, particularly preferably 6 to 12 moles of ethylene oxide to 1 mole of alkanol, which is preferably selected from caprylic alcohol, 2-ethylhexyl alcohol, capric alcohol, lauryl alcohol, isotridecyl alcohol, tridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and from their technical mixtures. Also adducts of 10-100 moles of ethylene oxide with technical fatty alcohols with 12-18 carbon atoms, such as coconut, palm, palm kernel or tallow fatty alcohol, are also suitable. Trideceth-6, Isotrideceth-6, Undeceth-6, Myreth-6, Laureth-10, Laureth-12, Laureth-15, Laureth-20, Laureth-30, Myreth-10, Myreth-12, Myreth-15, Myreth-20, Myreth-30, Ceteth-10, Ceteth-12, Ceteth-15, Ceteth-20, Ceteth-30, Steareth-10, Steareth-12, Steareth-15, Steareth-20, Steareth-30, Oleth-10, Oleth-12, Oleth-15, Oleth-20, Oleth-30, Ceteareth-10, Ceteareth-15, Ceteareth-12, Ceteareth-15, Ceteareth-20, Ceteareth-30 and Coceth-10, Coceth-12, Coceth-15, Coceth-20 and Coceth-30; Trideceth-6 and Isotrideceth-6 and mixtures thereof are particularly preferred.

The ethoxylated C₈-C₃₀ carboxylic acids have the formula R¹O(CH₂CH₂O)ₙH, where R¹O stands for a linear or branched saturated or unsaturated acyl radical with 8-30 carbon atoms and n, the average number of ethylene oxide units per molecule, is a number from 5-30, preferably 6-20, particularly preferably 6-12 moles of ethylene oxide with 1 mole of C₈-C₃₀ carboxylic acid, which is preferably selected from caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselinic acid, arachidic acid, gadoleic acid, behenic acid, erucic acid and brassidic acid and from their technical mixtures. Also adducts of 5 to 30, preferably 6 to 20, particularly preferably 6 to 12 moles of ethylene oxide with technical fatty acids with 12 to 18 carbon atoms, such as coconut, palm, palm kernel or tallow fatty acid, are suitable.

Agents which are extraordinarily preferred as contemplated herein contain at least one sodium polyacrylate with a weight-average molecular weight M_{w} in the range from about 1,000,000 to about 20,000,000 Daltons, preferably from about 6,000,000 to about 15,000,000 Daltons, the sodium polyacrylate being present in a water-in-oil emulsion is contained pre-gelled, said water-in-oil emulsion, in each case based on their total weight, from about 40-60% by weight sodium polyacrylate, a total of from about 25-45% by weight oil (s), preferably mineral oil, in total Contains from about 0.5-4.9% by weight of surfactant (s), preferably from about 0.5-4.9% by weight of non-ionic surfactant (s), and from about 0.5-4.9% by weight of water.

The agent as contemplated herein preferably contains sodium polyacrylate in a total amount of from about 0.1-3% by weight, preferably from about 0.3-2% by weight, preferably from about 0.6-1.1% by weight, based in each case on the weight of the product.

In a preferred embodiment, the agent as contemplated herein further contains at least one crosslinked copolymer of acrylic acid and non-ethoxylated esters of acrylic acid with linear C10-C30-monoalcohols (c).

(c) At Least One Cross-Linked Copolymer of Acrylic Acid and Non-Ethoxylated Esters of Acrylic Acid with Linear C10-C30-Monoalcohols

Agents preferred as contemplated herein contain at least one crosslinked copolymer composed of acrylic acid and non-ethoxylated esters of acrylic acid with linear C10-C30 monoalcohols as monomers. At least one crosslinked copolymer of acrylic acid and non-ethoxylated esters of acrylic acid with linear C10-C30 monoalcohols is preferably selected from copolymers with the INCI name acrylates/C10-30 alkyl acrylate cross polymer. Sucrose allyl ether or pentaerythrityl allyl ether is preferably contained as the crosslinking agent.

Crosslinked copolymers of acrylic acid and non-ethoxylated esters of acrylic acid with linear C10-C30 monoalcohols which are particularly preferred as contemplated herein can be obtained by polymerizing a monomer mixture which, based on its weight, is from about 80-99% by weight, preferably from about 90-98% by weight. %, acrylic acid, at least one non-ethoxylated ester of acrylic acid with linear C10-C30 monoalcohols in a total amount of from about 0.9-19.9% by weight, preferably from about 2-10% by weight, and at least one crosslinking agent in one contains a total of from about 0.1-4-% by weight.

Further crosslinked copolymers of acrylic acid and non-ethoxylated esters of acrylic acid with linear C10-C30 monoalcohols which are particularly preferred as contemplated herein about 0.5% by weight dispersion in water at about 25° C. and a pH in the range from about 5.8-6.3 has a viscosity in the range from about 45,000 to about 65,000 mPas, measured with a Brookfield RVF or Brookfield RVT viscometer at a rotation frequency of about 20 min⁻¹ with spindle 7.

The agent as contemplated herein preferably contains at least one crosslinked copolymer of acrylic acid and non-ethoxylated esters of acrylic acid with linear C10-C30-Monoalcohols in a total amount of from about 0.05-3% by weight, preferably from about 0.1-1.5% by weight. %, preferably from about 0.2-1% by weight, in each case based on the weight of the composition.

### (d) Surfactants

A surface-active substance is preferably additionally added to the agents as contemplated herein, such surface-active substances being referred to as surfactants or as emulsifiers, depending on the field of application: They are preferably selected from anionic, zwitterionic, amphoteric and non-ionic surfactants and emulsifiers.

For the purposes of the present application, surfactants and emulsifiers are amphiphilic (bifunctional) compounds which include at least one hydrophobic and at least one hydrophilic part of the molecule.

The hydrophobic radical is preferably a hydrocarbon chain with 8 to 30 carbon atoms, which can be saturated or unsaturated, linear or branched. This C₈-C₃₀ alkyl chain is particularly preferably linear. The basic properties of the surfactants and emulsifiers are oriented adsorption at interfaces as well as aggregation to micelles and the formation of lyotropic phases.

When selecting surfactants suitable as contemplated herein, it may be preferred to use a mixture of surfactants in order to optimally adjust the properties of the agents as contemplated herein.

Suitable anionic surfactants for the agents as contemplated herein are all anionic surfactants suitable for use on the human body, which have a water-solubilizing anionic group, for example a sulfate, sulfonate or phosphate group, and a lipophilic alkyl group with about 8 to 30 carbon atoms, preferably 8 to 24 carbon atoms in the molecule, with the exception of linear and branched fatty acids with 8 to 30 carbon atoms and their salts (soaps). In addition, glycol or polyglycol ether groups, ester, ether and amide and hydroxyl groups may also be present in the molecule. Examples of suitable anionic surfactants are, in each case in the form of the sodium, potassium and ammonium and the mono-, di- and trialkanolammonium salts with 2 to 4 carbon atoms in the alkanol group, polyethoxylated ether carboxylic acids, acyl sarcosines, acyl taurines, acyl isethionates, sulfosuccinic acid mono- and dialkyl esters and sulfosuccinic acid mono-alkyl polyoxyethyl esters with 1 to 6 ethylene oxide groups, linear alkane sulfonates, linear alpha-olefin sulfonates, sulfonates of unsaturated fatty acids with up to 6 double bonds, alpha-sulfo fatty acid methyl esters of fatty acids, C₈-C₂₀ alkyl sulfates and C₈-C₂₀ alkyl ether sulfate with 1 to 15 oxyethyl groups, mixtures of surface-active hydroxy sulfonates, sulphated hydroxyalkyl polyethylene and/or hydroxyalkylene propylene glycol ethers, esters of tartaric acid or citric acid with ethoxylated or propoxylated fatty alcohols, optionally polyethoxylated alkyl and/or alkenyl ether phosphates, sulfated fatty acid alkylene glycol esters and monoglyceride sulfates and monoglyceride ether sulfates. Preferred anionic surfactants are selected from C₈-C₂₀ alkyl sulfates, C₈-C₂₀ alkyl ether sulfates and C₈-C₂₀ ether carboxylic acids, each with 8 to 20 carbon atoms in the alkyl group and 0 to 12 ethylene oxide groups in the molecule. Sodium Laureth-2 sulfate is particularly preferred.

Zwitterionic surfactants are those surface-active compounds which carry a lipophilic alkyl group with about 8 to 30 C atoms, preferably 8 to 24 C atoms and at least one quaternary ammonium group and at least one carboxylate, sulfonate or sulfate group. Particularly suitable zwitterionic surfactants are the so-called betaines such as the N-alkyl-N, N-dimethylammonium glycinate, for example coco alkyl dimethylammonium glycinate, N-acyl aminopropyl-N, N-dimethylammonium glycinate, for example coco acyl aminopropyl dimethylammonium glycinate (with the INCI name Cocamidopropyl Betaine), and 2-alkyl-3-carboxymethyl-3-hydroxyethyl-imidazolines each having 8 to 18 carbon atoms in the alkyl or acyl group and coco acyl amino ethyl hydroxyethyl carboxymethyl glycinate. A preferred zwitterionic surfactant is the coco acyl aminopropyl dimethylammonium glycinate known under the INCI name Cocamidopropyl Betaine.

Amphoteric surfactants are surface-active compounds which contain a C₈-C₃₀ alkyl or acyl group and at least one free amino group and at least one —COOH— or —SO₃H group in the molecule and are capable of forming internal salts. Examples of suitable ampholytic surfactants are N-alkyl glycines, N-alkyl propionic acids, N-alkylaminobutyric acids, N-alkyliminodipropionic acids, N-hydroxyethyl-N-alkyl amidopropyl glycines, N-alkyl taurines, N-alkyl sarcosines, 2-alkylaminopropionic acids and alkylaminoacetic acids containing around 8 to 30 carbon atoms in the alkyl group. Particularly preferred amphoteric surfactants are N-coco alkyl amino propionate, coco acyl amino ethyl amino propionate and C₁₂-C₁₈ acyl sarcosine.

Anionic or zwitterionic surfactants preferred as contemplated herein are selected from C₈-C₂₀ alkyl sulfates, C₈-C₂₀ alkyl ether sulfates and C₈-C₂₀ ether carboxylic acids, each with 8 to 20 carbon atoms in the alkyl group and 0 to 12 ethylene oxide groups in the molecule, sodium Laureth-2 sulfate is particularly preferred, furthermore from coco acyl amino propyl dimethylammonium glycinate and from mixtures of these surfactants.

Agents which are extremely preferred as contemplated herein contain at least one anionic or zwitterionic surfactant selected from C₈-C₂₀ alkyl sulfates, C₈-C₂₀ alkyl ether sulfates and C₈-C₂₀ ether carboxylic acids, each with 8 to 20 C atoms in the alkyl group and 0 to 12 ethylene oxide groups in the molecule, with sodium Laureth-2 sulfate being particularly preferred, furthermore from coconut acyl amino propyl dimethylammonium glycinate and from mixtures of these surfactants.

If a surfactant is present in the compositions as contemplated herein, the compositions as contemplated herein contain at least one surfactant, preferably at least one anion or zwitterion surfactant, in a total amount of from about 0.3-5% by weight, preferably from about 0.5-4% by weight, each based on the weight of the product.

The agents as contemplated herein particularly preferably have a viscosity in the range of from about 8,000-100,000 mPas, preferably from about 10,000-80,000 mPas, preferably from about 12,000-60,000 mPas, in particular from about 13,000-50,000 mPas, in each case measured at about 20° C. using a Brookfield rotary viscometer at a rotation frequency from about 4 min−1 with spindle 5.

The agent as contemplated herein usually has a pH of about 2.5-11, measured at about 22° C.

Depending on the type of substantive dyes (non-ionic, cationic or anionic) to be used in the agent as contemplated herein, the pH is preferably adjusted to either acidic, neutral or basic.

In a preferred embodiment, the agent as contemplated herein has a pH in the range from about 3.0-11, particularly preferably from about 4.5-10.5, particularly preferably from about 5.5-10, in particular from about 6-9.5, measured in each case at about 22° C.

The present application also includes agents as contemplated herein which have a pH in the range from generally from about 3-11, preferably from about 3.5-9.5, preferably from about 4-8, in particular from about 5-7.5, measured in each case at about 22° C.

The pH value can be measured, for example, with a glass electrode, which is usually designed in the form of a combination electrode. The pH values of the present disclosure are pH values measured at a temperature of about 22° C.

To adjust the pH value, alkalizing agents or acidifying agents (e) can additionally be added to the agent as contemplated herein.

Suitable alkalizing agents are preferably selected from the group including ammonia, alkanolamines, basic amino acids and inorganic alkalizing agents. Preferred inorganic alkalizing agents are magnesium carbonate, sodium hydroxide, potassium hydroxide, sodium silicate and sodium metasilicate. Alkanolamines are preferably selected from monoethanolamine (in particular 2-aminoethanol), 2-amino-2-methylpropanol and triethanolamine. The basic amino acids which can be used as alkalizing agents are preferably selected from the group including arginine, lysine, ornithine and histidine, particularly preferable arginine.

Acidifying agents which can be used to adjust the pH are organic acids, such as citric acid, acetic acid, ascorbic acid, benzoic acid, lactic acid, malic acid and maleic acid, and mineral acids, such as hydrochloric acid, sulfuric acid or phosphoric acid.

If an alkalizing agent or acidifying agent is used in the agents as contemplated herein, the agent as contemplated herein contains at least one alkalizing agent or at least one acidifying agent in a total amount of from about 0.05-3% by weight, preferably from about 0.1-2% by weight, preferably from about 0.2-1.5% by weight, each based on the weight of the agent.

Furthermore, the agents as contemplated herein can contain further active ingredients, auxiliaries and additives.

Suitable active ingredients, auxiliaries and additives are, for example, oils; non-ionic polymers such as vinyl pyrrolidinone/vinyl acrylate copolymers, polyvinyl pyrrolidinone, vinyl pyrrolidinone/vinyl acetate copolymers, polyethylene glycols and polysiloxanes; additional silicones, such as volatile or non-volatile, straight-chain, branched or cyclic, cross-linked or non-cross-linked polyalkylsiloxanes (such as Dimethicone or Cyclomethicone), polyarylsiloxanes and/or polyalkylarylsiloxanes, in particular polysiloxanes with organofunctional groups, such as substituted or unsubstituted amines (Amodimethicone), carboxyl, alkoxy and/or hydroxyl groups (Dimethicone Copolyole), linear polysiloxaneA)-polyoxyalkyleneB) block copolymers, grafted silicone polymers; cationic polymers such as quaternized cellulose ethers, polysiloxanes with quaternary groups, dimethyl diallyl ammonium chloride polymers, acrylamide dimethyldiallyl ammonium chloride copolymers, dimethylamino ethylmethacrylate vinylpyrrolidinone copolymers quaternized with diethyl sulfate, vinyl pyrrolidinone imidazolinium methochloride copolymers and quaternized polyvinyl alcohol, zwitterionic and amphoteric polymers; hair conditioning compounds such as phospholipids, for example lecithin and cephalins; perfume oils, dimethyl isosorbide and cyclodextrins; active ingredients that improve fiber structure, in particular mono-, di- and oligosaccharides such as, for example, glucose, galactose, fructose, fructose and lactose; dyes for coloring the agent; anti-dandruff agents such as piroctone olamine, zinc omadine and climbazole; amino acids and oligopeptides; animal and/or vegetable-based protein hydrolyzates and in the form of their fatty acid condensation products or, where appropriate, anionically or cationically modified derivatives; light stabilizers and UV blockers; active ingredients such as panthenol, pantothenic acid, pantolactone, allantoin, pyrrolidinone carboxylic acids and their salts and bisabolol; polyphenols, especially hydroxycinnamic acids, 6,7-dihydroxycoumarins, hydroxybenzoic acids, catechins, tannins, leucoanthocyanidins, anthocyanidins, flavanones, flavones and flavonols; ceramides or pseudo-ceramides; vitamins, provitamins and vitamin precursors; plant extracts; waxes such as beeswax and montan wax; swelling and penetration substances such as glycerol, propylene glycol monoethyl ether, carbonate, hydrogen carbonate, guanidine, ureas and primary, secondary and tertiary phosphates; opacifiers such as latex, styrene/PVP and styrene/acrylamide copolymers; pearlescent agents, such as ethylene glycol mono- and distearate as well as PEG-3 distearate and pigments.

Oils which are particularly preferred as contemplated herein are selected from the esters of linear or branched saturated or unsaturated fatty alcohols having 2 to 30 carbon atoms with linear or branched saturated or unsaturated fatty acids having 2 to 30 carbon atoms, which may be hydroxylated. These include cetyl-2-ethylhexanoate, 2-hexyldecyl stearate, 2-hexyldecyl laurate, isodecyl neopentanoate, isononyl isononanoate, 2-ethylhexyl palmitate and 2-ethylhexyl stearate. Also preferred are isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid-2-butyl octanoate, diisotridecyl acetate, n-butyl stearate, n-hexyl laurate, n-decyl oleate, oleyl oleate, oleyl erucate, erucyl oleate, erucyl erucate, ethylene glycol dioleate and ethylene glycol dipalmitate.

Further oils preferred as contemplated herein are selected from natural and synthetic hydrocarbons, particularly preferably from mineral oils, paraffin oils, C₁₈-C30 isoparaffins, in particular isoeicosane, polyisobutenes and polydecenes, which are known, for example, under the name Emery® 3004, 3006, 3010 or under the name Ethylflo® from Albemarle or Nexbase® 2004G available from Nestle, further selected from C8-C16 isoparaffins, in particular from isodecane, isododecane, isotetradecane and isohexadecane and mixtures thereof, and 1,3-di-(2-ethylhexyl) cyclohexane.

Further oils preferred as contemplated herein are selected from the benzoic acid esters of linear or branched C₈-C₂₂ alkanols. Benzoic acid C₁₂-C₁₅ alkyl esters, benzoic acid isostearyl esters, ethylhexyl benzoate and octyl dodecyl benzoate are particularly preferred.

Further oils preferred as contemplated herein are selected from fatty alcohols having 6 to 30 carbon atoms, which are liquid at about 20° C. and can be unsaturated or branched and saturated or branched and unsaturated. Preferred alcohol oils are lauryl alcohol, a main component of the coconut fatty alcohol, furthermore 2-octyldodecanol, 2-hexyldecanol, 2-ethylhexyl alcohol and isostearyl alcohol.

Further preferred oils are selected from mixtures of guerbet alcohols and guerbet alcohol esters, e.g. mixtures of 2-hexyldecanol and 2-hexyldecyl laurate.

Further cosmetic oils preferred as contemplated herein are selected from the triglyceride's (=triple esters of glycerol) of linear or branched, saturated or unsaturated, optionally hydroxylated C₈₋₃₀-fatty acids. The use of natural oils, e.g. amaranth seed oil, apricot kernel oil, argan oil, avocado oil, babassu oil, cotton seed oil, borage seed oil, camelina oil, thistle oil, peanut oil, pomegranate kernel oil, grapefruit seed oil, hemp oil, hazelnut oil, elderberry seed oil, currant seed oil, jojoba oil, linseed oil, macadamia nut oil, corn germ oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn seed oil, sesame oil, soybean oil, sunflower oil, grape seed oil, walnut oil, wild rose oil, wheat germ oil, and the liquid portions of coconut oil and the like is particularly preferred. However, also synthetic triglyceride oils, in particular capric/caprylic triglycerides, for e.g. the commercial products Myritol® 318, Myritol® 331 (BASF) or Miglyol® 812 (Hills) with unbranched fatty acid residues and glyceryl triisostearin with branched fatty acid residues are preferred.

Further cosmetic oils which are particularly preferred as contemplated herein are selected from the dicarboxylic acid esters of linear or branched C₂-C₁₀ alkanols, in particular diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate and di-(2-hexyldecyl) succinate.

Further preferred cosmetic oils as contemplated herein are selected from the adducts of 1 to 5 propylene oxide units with mono- or polyvalent C₈-C₂₂ alkanols, such as octanol, decanol, decanediol, lauryl alcohol, myristyl alcohol and stearyl alcohol, e.g. PPG-2 myristyl ether and PPG-3 myristyl ether.

Further cosmetic oils preferred as contemplated herein are selected from the adducts of at least 6 ethylene oxide and/or propylene oxide units with mono- or polyvalent C3-C22 alkanols such as glycerol, butanol, butanediol, myristyl alcohol and stearyl alcohol, which may be esterified if desired, e.g. PPG-14 butyl ether, PPG-9 butyl ether, PPG-10 butanediol, PPG-15 stearyl ether and glycereth-7-diisononanoate.

Further preferred cosmetic oils as contemplated herein are selected from the C₈-C₂₂ fatty alcohol esters of monovalent or polyvalent C₂-C₇ hydroxycarboxylic acids, in particular the esters of glycolic acid, lactic acid, malic acid, tartaric acid, citric acid and salicylic acid.

Further cosmetic oils preferred as contemplated herein are selected from the symmetrical, asymmetrical or cyclic esters of carbonic acid with C₃-C₂₂ alkanols, C₃-C₂₂ alkanediols or C₃-C₂₂ alkanediols, e.g. dicaprylyl carbonate or the esters according to the teaching of DE 19756454 A1, in particular glycerol carbonate.

Further cosmetic oils, which may be preferred as contemplated herein, are selected from the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols.

Further cosmetic oils which are suitable as contemplated herein are selected from the silicone oils, which include e.g. dialkyl- and alkyl aryl siloxanes, such as cyclopentasiloxane, cyclohexasiloxane, dimethylpolysiloxane and methylphenylpolysiloxane, but also hexamethyldisiloxane, octamethyltrisiloxane and decamethyltetrasiloxane. Volatile silicone oils may be preferred, which can be cyclic, such as octamethylcyclotetrasiloxane, decamethylcyclopentasiloxane and dodecamethylcyclohexasiloxane, as well as mixtures thereof, as, for example, contained in the commercial products DC 244, 245, 344 and 345 from Dow Corning. Also suitable are volatile linear silicone oils, in particular hexamethyldisiloxane (L₂), octamethyltrisiloxane (L₃), decamethyltetrasiloxane (L₄) and any two- and three-component mixtures of L₂, L₃ and/or L₄, preferably mixtures such as those included, for example, in the commercial products DC 2-1184, Dow Corning® 200 (0.65 cSt) and Dow Corning® 200 (1.5 cSt) Dow Corning. Preferred non-volatile silicone oils are selected from higher molecular weight linear dimethylpolysiloxanes, commercially available e.g. under the name Dow Corning® 190, Dow Corning® 200 Fluid with kinematic viscosities (about 25° C.) in the range of 5-100 cSt, preferably 5-50 cSt or also 5-10 cSt, and dimethylpolysiloxane with a kinematic viscosity (about 25° C.) of 350 cSt.

As contemplated herein, it can be extremely preferred to use mixtures of the aforementioned oils.

Preferred colorants as contemplated herein cosmetic oil is selected from natural and synthetic hydrocarbons, particularly preferably from paraffin oils, C₁₈-C₃₀-Isoparaffins, in particular isoeicosane, polyisobutenes and polydecenes, C₈-C₁₆ isoparaffins, and 1,3-di-(2-ethylhexyl) cyclohexane; the benzoic acid esters of linear or branched C₈-C₂₂ alkanols; fatty alcohols with 6 to 30 carbon atoms, which are liquid at about 20° C. and 1013 mbar and can be unsaturated or branched and saturated or branched and unsaturated, preferably lauryl alcohol and 2-octyldodecanol; triglycerides of linear or branched, saturated or unsaturated, optionally hydroxylated C₈-C₃₀ fatty acids, in particular natural oils; dicarboxylic acid esters of linear or branched C₂-C₁₀ alkanols; esters of linear or branched saturated or unsaturated fatty alcohols with 2 to 30 carbon atoms with linear or branched saturated or unsaturated fatty acids with 2 to 30 carbon atoms, which may be hydroxylated; adducts of 1 to 5 propylene oxide units with mono- or polyvalent C₈-C₂₂ alkanols; adducts of at least 6 ethylene oxide and/or propylene oxide units onto mono- or polyvalent C₃-C₂₂ alkanols; C₈-C₂₂ fatty alcohol esters of monovalent or polyvalent C₂-C₇ hydroxycarboxylic acids; symmetrical, asymmetrical or cyclic esters of carbonic acid with C₃-C₂₂ alkanols, C₃-C₂₂ alkane diols or C₃₋₂₂ alkane triols; the esters of dimers of unsaturated C₁₂-C₂₂ fatty acids (dimer fatty acids) with monovalent linear, branched or cyclic C₂-C₁₈ alkanols or with polyvalent linear or branched C₂-C₆ alkanols; silicone oils and mixtures of the aforementioned substances.

Further preferred non-oxidative colorants as contemplated herein are exemplified in a way that based in each case on the weight of the colorant, at least one oil in a total amount of from about 0.1-20% by weight, preferably from about 0.5-10% by weight, particularly preferably from about 1 to about 6% by weight is contained.

Further preferred non-oxidative colorants as contemplated herein contain at least one C₁₄-C₃₀ fatty alcohol which is solid at about 20° C. and 1013 mbar.

Preferred C₁₄-C₃₀ fatty alcohols, which are solid at about 20° C. and 1013 mbar, are selected from tetradecan-1-ol (tetradecyl alcohol, myristyl alcohol), hexadecan-1-ol (hexadecyl alcohol, cetyl alcohol), octadecan-1-ol (octadecyl alcohol, stearyl alcohol), arachyl alcohol (eicosan-1-ol), heneicosyl alcohol (heneicosan-1-ol) and/or behenyl alcohol (docosan-1-ol) and mixtures thereof.

Further preferred non-oxidative colorants as contemplated herein, based in each case on the weight of the colorant, they contain at least one C₁₄-C₃₀ fatty alcohol, which is solid at about 20° C. and 1013 mbar, in a total amount of about 0.1-10% by weight, preferably from about 0.3-5% by weight, particularly preferably from about 0.5-2% by weight.

The expert will select these other active ingredients, auxiliaries and additives in accordance with the desired properties of the agents.

The active ingredients, auxiliaries and additives mentioned above in addition to the oils and solid C₁₄-C₃₀-fatty alcohols can be included, if they are used in the agents as contemplated herein, preferably in amounts of from about 0.001-25% by weight, in particular from about 0.1-15% by weight, based on the weight of the colorant.

Further non-oxidative colorants preferred as contemplated herein are exemplified by a water content of from about 70-95% by weight, preferably from about 78-91% by weight, particularly preferably from about 80-86% by weight, in each case based on the weight of the colorant.

The agent as contemplated herein can in principle be present in the form of creams, emulsions, gels or surfactant-containing foaming solutions, such as shampoos, foam aerosols, foam formulations or other preparations, which are suitable for use on the hair.

The agent as contemplated herein is preferably in the form of a cream, an emulsion or a gel.

The agent as contemplated herein is exemplified by good durability on keratin fibers even after several washes, simple application and generally short exposure times.

In general, the shelf life of the dye of the agent as contemplated herein on the keratin fibers, in particular human hair, is from about 1 to about 24 washes, preferably from about 2 to about 14 washes, at from about 10-50° C., preferably at from about 25-40° C. for from about 5 minutes to about 2 Hours.

Another object of the present application is a method for the non-oxidative, preferably semi-permanent, coloring of keratin fibers, in particular human hair, the agent as contemplated herein being applied directly to the keratin fibers in the form of a one-component agent, the method is preferably carried out having an exposure time of the keratin fibers from about 5 seconds to about 45 minutes, preferably from about 1 to about 40 minutes, particularly preferably from about 5 to about 30 minutes, extremely preferably from about 10 to about 20 minutes, at room temperature and/or at from about 30-60° C., preferably at from about 32-50° C.

As contemplated herein, the term “room temperature” denotes the temperature in the room in which a person usually uses a hair dye, that is to say usually a bathroom or a hairdressing salon, where a temperature in the range from about 10-29° C. prevails.

The agent as contemplated herein can also act at least about 30° C., preferably at from about 30-60° C., particularly preferably at from about 32-50° C., if the hair is heated, for example, with a heat hood or with a heat radiator.

For the dyeing method as contemplated herein and for the dyeing method preferred as contemplated herein, what has been said above regarding the non-oxidative coloring agents as contemplated herein and preferred as contemplated herein applies mutatis mutandis.

In summary, the subject matter of the present disclosure relates to the following particularly preferred embodiments:

Agent for dyeing keratin fibers, in particular human hair, contained in a cosmetic carrier
(a) at least one substantive dye and
(b) sodium polyacrylate, preferably with a weight average molecular weight M_{w} in the range from about 1,000,000 to about 20,000,000 Daltons, preferably from about 6,000,000 to about 15,000,000 Daltons.
   Agent for dyeing keratin fibers, in particular human hair, contained in a cosmetic carrier
(a) at least one substantive dye and
(b) sodium polyacrylate, preferably with a weight average molecular weight M_{w} in the range from about 1,000,000 to about 20,000,000 Daltons, preferably from about 6,000,000 to about 15,000,000 Daltons, contains at least one non-ionic direct dye.

Agent for dyeing keratin fibers, in particular human hair, contained in a cosmetic carrier
(a) at least one substantive dye and
(b) Sodium polyacrylate, preferably with a weight-average molecular weight M_{w} in the range from about 1,000,000 to about 20,000,000 Daltons, preferably from about 6,000,000 to about 15,000,000 Daltons, contains at least one anionic direct dye.

Agent for dyeing keratin fibers, in particular human hair, contained in a cosmetic carrier
(a) at least one substantive dye and
(b) sodium polyacrylate, preferably with a weight-average molecular weight M_{w} in the range from about 1,000,000 to about 20,000,000 Daltons, preferably from about 6,000,000 to about 15,000,000 Daltons, contains at least one cationic direct dye.

Agent for dyeing keratin fibers, in particular human hair, contained in a cosmetic carrier
(a) at least one substantive dye and
(b) sodium polyacrylate, preferably with a weight average molecular weight M_{w} in the range from about 1,000,000 to about 20,000,000 Daltons, preferably from about 6,000,000 to about 15,000,000 Daltons, sodium polyacrylate is used as pre-gelled in a water-in-oil emulsion, said water-in-oil emulsion being preferred, in each case based on their weight, from about 40-60% by weight sodium polyacrylate, in total from about 25-45% by weight oil (s), in total from about 0.5-4.9% by weight % Surfactant (s), preferably from about 0.5-4.9% by weight non-ionic surfactant (s), and from about 0.5-4.9% by weight water

Agent for dyeing keratin fibers, in particular human hair, contained in a cosmetic carrier
(a) at least one substantive dye and
(b) sodium polyacrylate, preferably with a weight-average molecular weight M_{w} in the range from about 1,000,000 to about 20,000,000 Daltons, preferably from about 6,000,000 to about 15,000,000 Daltons, at least one crosslinked copolymer of acrylic acid and non-ethoxylated esters of acrylic acid with linear C₁₀-C₃₀-monoalcohols (c), preferably in a total amount of from about 0.05-3% by weight, preferably from about 0.1-1.5% by weight, preferably from about 0.2-1% by weight, in each case based on the weight of the agent, which is preferably selected from copolymers with the INCI name acrylates/C10-30 alkyl acrylate cross polymer.

Agent for dyeing keratin fibers, in particular human hair, contained in a cosmetic carrier
(a) at least one substantive dye and
(b) sodium polyacrylate, preferably with a weight-average molecular weight M_{w} in the range from about 1,000,000 to about 20,000,000 Daltons, preferably from about 6,000,000 to about 15,000,000 Daltons, exemplified by a viscosity in the range from about 8,000 to about 100,000 mPas, preferably from about 10,000 to about 80,000 mPas, preferably from about 12,000-60,000 mPas, in particular from about 13,000-50,000 mPas, each measured at about 20° C. with a Brookfield rotation viscometer at a rotation frequency of from about 4 min−1 with spindle 5.

Agent for dyeing keratin fibers, in particular human hair, contained in a cosmetic carrier
(a) at least one substantive dye and
(b) sodium polyacrylate, preferably with a weight-average molecular weight M_{w} in the range from about 1,000,000 to about 20,000,000 Daltons, preferably from about 6,000,000 to about 15,000,000 Daltons, at least one surface-active substance (d), preferably selected from the group including anionic surfactants, zwitterionic surfactants, amphoteric surfactants, non-ionic surfactants and emulsifiers.

Agent for dyeing keratin fibers, in particular human hair, contained in a cosmetic carrier
(a) at least one substantive dye and
(b) sodium polyacrylate, preferably with a weight-average molecular weight M_{w} in the range from about 1,000,000 to about 20,000,000 Daltons, preferably from about 6,000,000 to about 15,000,000 Daltons, is in the form of a cream, an emulsion or a gel.

The following examples are intended to illustrate the subject matter of the present disclosure, without restricting it thereto.

### EXAMPLES

TABLE 1
Colorant A as contemplated herein

| | Sample weight |
|---|---|
| Ingredient | (wt.-%) |
| Acrylates/C10-30 alkyl acrylate cross polymer | 0.40 |
| Paraffinum Liquidum (ex ¹⁾) | 0.74 |
| C11-13 Isoparaffin (ex ¹⁾) | 0.06 |
| Trideceth-6 (ex ¹⁾) | 0.1 |
| Sodium polyacrylate (ex ¹⁾) | 1.00 |
| 2-Octyldodecanol | 4.00 |
| Coconut fat alcohol, C12-18 | 0.75 |
| Fatty alcohol-EO sulphate-Na C12-14 2EO | 3.00 |
| Monoethanolamine (2-aminoethan-1-ol) | 1.14 |
| HC Red 3²⁾ | 2.00 |
| Water | ad 100.00 |

¹⁾ Flocare ® DP/ES-502¹: Water-in-oil emulsion made from sodium polyacrylate, mineral oil, trideceth-6, C11-13 isoparaffin and water
²⁾non-ionic direct dye

TABLE 2
Colorant B as contemplated herein

| | Sample weight |
|---|---|
| Ingredient | (wt.-%) |
| Acrylates/C10-30 alkyl acrylate cross polymer | 0.40 |
| Sodium polyacrylate | (active content) 1.00 |
| Mineral oil | 0.74 |
| Trideceth-6 | 0.10 |
| C11-13 Isoparaffin | 0.06 |
| 2-Octyldodecanol | 4.00 |
| Fatty alcohol, C12-18 | 0.75 |
| Fatty alcohol-EO sulphate-Na C12-14 2EO | 0.81 |
| Monoethanolamine (2-aminoethan-1-ol) | 1.13 |
| HC Red 3²⁾ | 2.00 |
| Water | ad 100.00 |

²⁾non-ionic direct dye

TABLE 3
Comparative dye C

| | Sample weight |
|---|---|
| Ingredient | (wt.-%) |
| Lorol ® ¹⁾ | 4.00 |
| Lanette ® D ²⁾ | 6.00 |
| Propyl paraben | 0.18 |
| Methyl paraben | 0.40 |
| Ceteareth-20 ³⁾ | 0.90 |
| Mackam ® 2CSF-40CG ⁴⁾ | 1.80 |
| HC Blue 12 ⁵⁾ | 0.05 |
| 4-Amino-3-nitrophenol | 1.00 |
| Polyethylene Glycol MG400 | 5.00 |
| Polyquaternium-6 | 0.50 |
| Nicotinamide | 0.15 |
| D-Panthenol 75% | 0.20 |
| Perfume | 0.30 |
| Water | ad 100.00 |

¹⁾ Fatty alcohol, C12-18
²⁾ Cetearyl alcohol
³⁾ Polyethylene glycol ether of cetearyl alcohol (20 units on average —CH2—CH2—O—)
⁴⁾ INCI Name: disodium cocoamphodipropionate
⁵⁾ non-ionic direct dye

### Example 1: Time Saving—Production (2 kg Batch Size)

| | Time required | |
|---|---|---|
| | for dye A as | Time spent |
| | contemplated | comparing |
| Production step | herein | dye C |
| Melting of the fat components (80° C.) | not applicable | 60 Min. |
| Incorporation of Flocare ® DP/ES-502 | 10 Min. | not applicable |
| Dissolve and add the dyes | 10 Min. | 10 Min. |
| Pre-emulsify and cool to approx. 45° C. | not applicable | 20 Min. |
| Add active ingredients and perfume | 5 Min. | 5 Min. |
| Cool the batch to approx. 30° C. | not applicable | 10 Min. |
| TOTAL | 25 Min. | 105 Min. |

### Example 2: Water Saving—Production (2 kg Batch Size)

It was cooled with running water. The longer the cooling times for a batch, the higher the consumption of cooling water and energy.
From example 1, the elimination of the cooling times results in great water and energy savings.
Cooling time—Production of the colorant A as contemplated herein: 0 Min.
Cooling time—Production of the comparative dye C: 30 Min.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A non-oxidative agent for dyeing keratin fibers included in a cosmetic carrier, the agent comprising:
(a) at least one substantive dye; and
(b) sodium polyacrylate.

2. The agent according to claim 1, further comprising at least one non-ionic direct dye.

3. The agent according to claim 1, further comprising at least one anionic direct dye.

4. The agent according to claim 1, further comprising at least one cationic direct dye.

5. The agent according to claim 1, wherein at least one substantive dye is present in a total amount of from about 0.001 to about 7% by weight, based on the total weight of the agent.

6. The agent according to claim 1, wherein the sodium polyacrylate is used as pre-gelled in a water-in-oil emulsion.

7. The agent according to claim 1, wherein the sodium polyacrylate is present in a total amount of from about 0.1 to about 3% by weight, based on the weight of the composition.

8. The agent according to claim 1, further comprising at least one crosslinked copolymer of acrylic acid and non-ethoxylated esters of acrylic acid with linear C10-C30-monoalcohols.

9. The agent according to claim 1, wherein the agent has a viscosity of from about 8,000 to about 100,000 mPas measured at 20° C. using a Brookfield rotary viscometer at a rotation frequency of 4 min−1 with spindle 5.

10. The agent according to claim 1, wherein the agent has a pH of from about 3.0 to about 11, measured at 22° C.

11. The agent according to claim 1, further comprising at least one surface-active substance.

12. The agent according to claim 1, further comprising at least one anionic or zwitterionic surfactant.

13. The agent according to claim 1, wherein the agent is from about 70 to about 95% by weight water.

14. The agent according to claim 1, further comprising at least one oil and/or at least one C₁₄-C₃₀ fatty alcohol which is solid at 20° C. and 1013 mbar.

15. A process for the non-oxidative coloring of keratin fibers, the process comprising applying the agent according to claim 1 directly to the keratin fibers in the form of a one-component agent.

16. The agent of claim 1, wherein:
the sodium polyacrylate has a weight average molecular weight M_{w} of from about 1,000,000 to about 20,000,000 Daltons;
the sodium polyacrylate is present in a total amount of from about 0.3 to about 2% by weight, based on the weight of the composition; and
the at least one substantive dye is present in a total amount of from about 0.5 to about 2% by weight, based on the total weight of the agent.

17. The agent of claim 1, wherein:
the sodium polyacrylate has a weight average molecular weight M_{w} of from about 6,000,000 to about 15,000,000 Daltons;
the sodium polyacrylate is present in a total amount of from about 0.6 to about 1.1% by weight, based on the weight of the composition; and
the at least one substantive dye is present in a total amount of from about 0.7 to about 1.2% by weight, based on the total weight of the agent.

18. The agent of claim 1, further comprising a crosslinked copolymer selected from copolymers with the INCI name acrylates/C10-30 alkyl acrylate cross polymer in a total amount of from about 0.05 to about 3 wt. %, based on the weight of the agent.

19. The agent of claim 1, further comprising a crosslinked copolymer selected from copolymers with the INCI name acrylates/C10-30 alkyl acrylate cross polymer in a total amount of from about 0.2 to about 1 wt. %, based on the weight of the agent.

20. The agent of claim 1, wherein the agent has a pH of from about 6.0 to about 9.5, measured at 22° C.
